(19) United States
(12) Patent Application Publication
(10) Pub. No.: US 2011/0120470 A1
(43) Pub. Date: May 26, 2011

(54) TREATMENT PROTOCOL TEMPLATE GENERATION AND BRANCHING LOGIC SYSTEM

(75) Inventor: Jonathan William Bowerbank, Thousand Oaks, CA (US)

(73) Assignee: HEALTHCARE CLINICAL CONSULTANTS, INC. DBA THERONYX, Thousand Oaks, CA (US)

(21) Appl. No.: 12/952,062

(22) Filed: Nov. 22, 2010

Related U.S. Application Data
(60) Provisional application No. 61/263,760, filed on Nov. 23, 2009.

(51) Int. Cl. A61M 16/00

(52) U.S. Cl. 128/204.23; 128/204.21

(57) ABSTRACT

A method for determining a next protocol step in a patient treatment protocol upon completion of a current treatment protocol step. The method includes accessing a treatment protocol template associated with a treatment protocol step. The treatment protocol step includes rules that determine when the protocol step is passed or failed and includes a first clinical decision point that specifies a second protocol step that is be performed next when the rules are passed and a second clinical decision point that specifies a third protocol step that is to be performed next when the rules are not passed. The method includes receiving input into parameter fields of the treatment protocol template, determining if the rules associated with the parameter fields of the treatment protocol template have been passed or not passed, and automatically determining which of the second or third protocol steps is to be performed next.

### CROSS-REFERENCE TO RELATED APPLICATIONS

This application claims the benefit of and priority to U.S. Provisional Patent Application Ser. No. 61/263,760 filed on Nov. 23, 2009, which application is incorporated herein by reference in its entirety.

### BACKGROUND

Hospitals and medical providers will often devise treatment protocols that involve treatment of a patient using a medical device. The actual treatment, however, is often performed by a technician or other medical professional other than the provider who devised the treatment protocol. This can lead to difficulties for both the technician and the medical provider.

For example, the treatment protocols often consist of several pages of instructions that are difficult for the technician to understand and follow. If one step written on a first page in the treatment protocol requires that the technician access a step written on a second page, problems may arise if the technician cannot properly follow or find the next step.

For the medical provider, problems arise when he or she is not able to ascertain if the technician complied with the treatment protocol in an acceptable manner. Further, if the technician deviates from the treatment provider, there may be no easy way for the medical provider to ascertain this fact.

### BRIEF SUMMARY

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One embodiment disclosed herein relates to a method for determining a next protocol step that is to be performed in a patient treatment protocol upon completion of a current treatment protocol step. The method includes accessing a treatment protocol template. The treatment protocol template is associated with a treatment protocol step of an underlying treatment protocol. The treatment protocol step includes rules that determine when the protocol step is passed or failed. The treatment protocol step includes a first clinical decision point that specifies a second protocol step that is to be performed next when the rules are passed and includes a second clinical decision point that specifies a third protocol step that is to be performed next when the rules are not passed. The treatment template may specify parameter fields that are associated with one or more of the rules of the treatment protocol step. The method also includes receiving input into the parameter fields of the treatment protocol template. The method further includes determining, based on the received input, if the rules associated with the parameter fields of the treatment protocol template have been successfully passed or unsuccessfully not passed. The method further includes, in response to determining if the rules associated with the parameter fields have passed or not passed, automatically determining which of the second or third protocol steps is to be performed next.

Another embodiment disclosed herein relates to a method for linking a next treatment protocol step with a protocol treatment template of a current treatment protocol step. The method includes accessing a treatment protocol step of an underlying patient treatment protocol for patient treatment using a medical device. The treatment protocol step includes one or more rules that are to be complied with in order to successfully perform the treatment protocol step. The method also includes generating a treatment protocol template for the treatment protocol step, the treatment protocol template including parameter fields associated with the one or more rules of the treatment protocol step. The method further includes linking the treatment protocol step and the generated treatment protocol template to a first clinical decision point that specifies a second protocol step that is to be performed next when the one or more rules are successfully passed. The method also includes linking the treatment protocol step and the generated treatment protocol templates to a second clinical decision point that specifies a third protocol step that is to be performed next when the one or more rules are not successfully passed.

Another embodiment disclosed herein relates to a computer system. The computing system includes a first module configured to access a patient treatment protocol, the patient treatment protocol including a plurality of clinical decision points that include one or more rules that are to be complied with in order to successfully treat a patient using a medical device that is associated with the patient treatment protocol; a treatment protocol template generation module configured to generate a treatment protocol template for each of the clinical decision points, each treatment protocol template specifying the one or more rules of the corresponding clinical decision point; a next step logic module configured to determine a second protocol template that is to be accessed and followed when the one or more rules of a specific generated treatment protocol template are successfully passed and to determine a third protocol template that is to be accessed and followed when the one or more rules of the specific generated treatment protocol template are not successfully passed; and one or more input modules for receiving information related to the specific generated treatment protocol template.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates a block diagram of a system in which the embodiments disclosed herein may be practiced;

FIG. 2 illustrates a block diagram of an embodiment of a protocol template generation and branching logic system in accordance with the embodiments disclosed herein;

FIG. 3 illustrates an example treatment protocol for weaning a patient off of a ventilator in accordance with the embodiments disclosed herein;

FIG. 4 illustrates a specific embodiment of the protocol template generation and branching logic system generating a treatment protocol template;

FIG. 5 illustrates a specific embodiment of the protocol template generation and branching logic system generating a treatment protocol template;

FIG. 6 illustrates a specific embodiment of the protocol template generation and branching logic system generating a treatment protocol template;

FIG. 7 illustrates a specific embodiment of the protocol template generation and branching logic system generating a treatment protocol template;

FIG. 8 illustrates a specific embodiment of the protocol template generation and branching logic system generating a treatment protocol template;

FIG. 9 illustrates a specific embodiment of the protocol template generation and branching logic system associating treatment templates with a protocol and determining the next step in a treatment protocol;

FIG. 10 illustrates a specific embodiment of the protocol template generation and branching logic system associating treatment templates with a protocol and determining the next step in a treatment protocol;

FIG. 11 illustrates a specific embodiment of the protocol template generation and branching logic system associating treatment templates with a protocol and determining the next step in a treatment protocol;

FIG. 12 illustrates a specific embodiment of the protocol template generation and branching logic system associating treatment templates with a protocol and determining the next step in a treatment protocol;

FIG. 13 illustrates a specific embodiment of the protocol template generation and branching logic system associating treatment templates with a protocol and determining the next step in a treatment protocol;

FIG. 14 illustrates a treatment protocol template in accordance with the embodiments disclosed herein;

FIG. 15 illustrates a specific embodiment of the protocol template generation and branching logic system showing what the next step should be;

FIG. 16 illustrates a treatment protocol template for a medical device in accordance with the embodiments disclosed herein;

FIG. 17 illustrates a flow chart of a method for determining a next step that is to be performed in a patient treatment protocol upon completion of a current treatment protocol step;

FIG. 18 illustrates a method 1800 for linking a next treatment protocol step with a protocol treatment template of a current treatment protocol step; and

FIG. 19 illustrates an example computing system for implementing the embodiments disclosed herein.

### DETAILED DESCRIPTION

The embodiments disclosed herein relate to a next treatment protocol step that is to be performed in a patient treatment protocol upon completion of a current treatment protocol step. The method includes accessing a treatment protocol template that is associated with a treatment protocol step. The method also includes electronically receiving or manually inputting clinical and diagnostic information into parameter fields with rules, all associated with a treatment protocol template which is further associated with a protocol step within a treatment protocol. The method further includes determining, based on the entered clinical and diagnostic information, if the rules, associated with the parameters of the treatment protocol template, have been successfully met (passed) or unsuccessfully met (failed). The method further includes, automatically determining from first treatment protocol step, a second treatment protocol step that is to be performed next, based on the parameter rules of the first protocol treatment template, associated with the first treatment protocol step, being successfully or unsuccessfully met. A method within each treatment protocol step creates two clinical decision points. One clinical decision point is referred to as the “On Pass Step” and is independently associated/linked with its own treatment protocol step. In the event that the rules associated with the parameters in a prior protocol template/protocol step were successfully met (passed), the program recommends the treatment protocol step that is associated with the “On Pass” clinical decision point. The same method is employed for the second clinical decision point associated within a treatment protocol step. This clinical decision point is referred to as the “On Fail Step”. Each time a clinician performs a treatment protocol step by entering clinical and diagnostic information in the associated treatment protocol template and the template data is programmatically evaluated, the program executes the methodology listed above to determine the next step that is to be performed by the clinician until all of the configured steps have been performed.

Reference will now be made to the figures wherein like structures will be provided with like reference designations. It is understood that the figures are diagrammatic and schematic representations of some embodiments of the invention, and are not limiting of the present invention, nor are they necessarily drawn to scale. It will be appreciated that the references to a first, second, etc element in the specification and claims, for example a second protocol treatment step, is not meant to imply sequential ordering unless explicitly states, but is instead meant to distinguish the elements from each other.

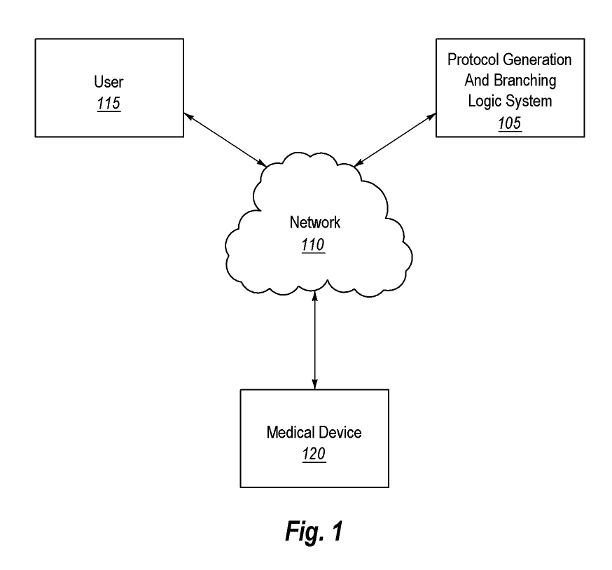

FIG. 1 illustrates a block diagram in which embodiments disclosed herein may be practiced. The block diagram includes a treatment protocol template generation and branching logic system 105. In some embodiments, the protocol template generation and branching logic system 105 can be located on a server. A server is a computer that provides services used by other computers. For example, a web server serves up web pages. Services or documents can be supplied centrally by the use of a server. In other embodiments, the services of the protocol template generation and branching logic system 105 can be distributed across multiple computing systems and shared peer-to-peer or through any other method. In further embodiments, the protocol template generation and branching logic system 105 may be implemented by the resources of a cloud computing system.

The protocol template generation and branching logic system 105 may be a hardware module, a software module, or any combination of the two that is configured to receive as input various treatment protocols and rules relating to a medical procedure. The medical procedure may involve patient treatment using a specific medical device. In one specific embodiment, the medical device may be a ventilator and the treatment protocols may be related to weaning a patient off of a ventilator. The protocol template generation and branching logic system 105 may also receive operational information from the medical device that is hooked up to a patient such as a ventilator. The protocol template generation and branching logic system 105 may then produce various web based and other types of treatment templates that provide easy instruction for a clinician as well as verification and other monitoring as will be described in more detail to follow. Further, the protocol template generation and branching logic system 105 provides branching logic that informs the clinician if a step in a protocol has been successful or unsuccessful and what the next step in the protocol should be when the current step is successful or when the current step is unsuccessful.

The protocol template generation and branching logic system 105 may be connected to a network 110. A computer network is a group of interconnected computers. The network 110 exemplarily includes the Internet, comprising a global internetwork formed by logical and physical connections between multiple wide area networks and/or local area networks and can optionally include the World Wide Web (“Web”), comprising a system of interlinked hypertext documents accessed via the Internet. Alternately or additionally, the network 110 includes one or more cellular RF networks and/or one or more wired and/or wireless networks such as, but not limited to, 802.xx networks, Bluetooth access points, wireless access points, IP-based networks, or the like. The network 110 may also include servers that enable one type of network to interface with another type of network. In one embodiment, the network 110 may be a hospital or other care facility network.

As also illustrated, a medical device 120 may be connected to the network 110. In operation, the medical device 120 provides medical care or other services to a patient that is connected to the medical device. The medical device 120 provides information to the protocol template generation and branching logic system 105 as will be explained in more detail to follow. The medical device 120 may be any reasonable medical device used in the medical industry to treat a patient. In one specific embodiment, the medical device 120 may be a ventilator that is used to help a patient breathe. In other embodiments, the medical device 120 may be, but is not limited to, a heart monitor, an EKG machine, an EEG machine, an X-ray machine, an ultrasound machine, or any other type of medical device that may be used in a treatment protocol to treat a patient. It will be appreciated that the medical device 120 may also include a combination of two or more medical devices as circumstances warrant.

The network 110 allows access to the protocol template generation and branching logic system 105 to a user 115. In some embodiments, the user 115 can be a treatment provider. Treatment provider, as used herein, is any person or entity which is authorized to perform medical treatment on a patient through use of the medical device 120. For example, the user 115 could be a physician or doctor, a nurse, a physician's assistant, a ventilator clinician or therapist, or the like. The user 115 may access the protocol template generation and branching logic system 105 locally or remotely using a user interface of a computing system. Thus, the user 115 need not be in the same location as the medical device 120.

The user 115 can utilize the functionality of the protocol template generation and branching logic system 105 to help evaluate and treat the patient connected to the medical device 120. This may be accomplished through use of the web based treatment templates as will be explained.

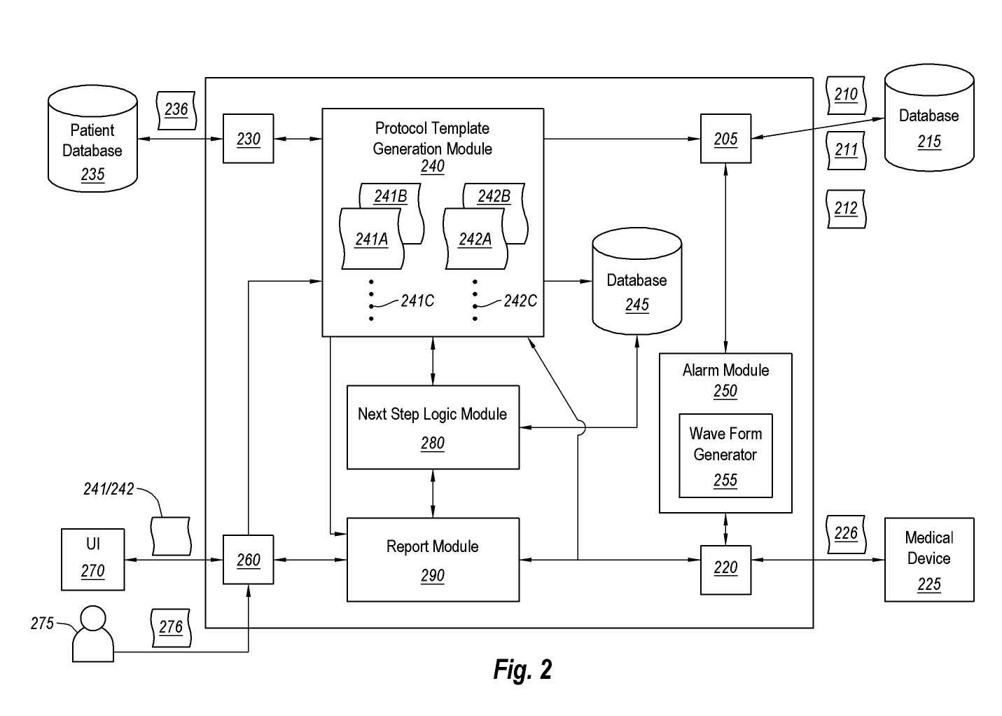

Attention is now made to FIG. 2, which illustrates a more detailed description of an embodiment of a protocol template generation and branching logic system 200, which may correspond to the protocol template generation and branching logic system 105 of FIG. 1. The system 200 can be implemented in hardware, software or any combination thereof. If the system 200 is implemented in software, the modules of the system 200 are stored in a computer-readable medium, to be accessed as needed to perform their functions. Additionally, if the system 200 is implemented in software, the tasks assigned to each module can be carried out by a processor, field-programmable gate array (FPGA) or any other logic device capable of carrying out software instructions or other logic functions.

It is understood that although the system 200 of FIG. 2 illustrates different modules performing different functions, the number of modules and their function can be changed without departing from the spirit or essential characteristics of the embodiments disclosed herein. That is, the functions of the modules may be combined or separated without restriction and without departing from the inventive concepts presented herein. Further, it will be appreciated that the system 200 may be implemented on a single computing system or it may be distributed over several different computing systems.

The protocol template generation and branching logic system 200 includes a treatment protocol retrieval module 205. The treatment protocol retrieval module 205 is configured to retrieve treatment protocols from a database 215. The treatment protocols contain treatment guidelines or rules for the treatment of patients that are currently connected to the medical device 120. In one embodiment, the guidelines or rules may be for weaning a patient off of a ventilator. Additionally, in some embodiments, the treatment protocols can include the date that the guidelines were produced and where the information was obtained.

In some embodiments, the treatment protocols include various treatment protocol steps (also referred herein simply as protocol steps) that each has associated rules that should be met or passed in order to successfully perform the treatment protocol step. In addition, each treatment protocol step will typically include two independent clinical decision points or treatment pathways. The first clinical decision point or treatment pathway, referred to previously as the “On Pass” step, links or points to another treatment protocol step that is performed next when the rules of the treatment protocol step are successfully passed or met. Likewise, the second clinical decision point or treatment pathway, referred to previously as the “On Fail” step, links or points to another treatment protocol step which is different from the “On Pass” step that is performed next when the rules of the treatment protocol step are not successfully passed or met.

For example, a first treatment protocol 210 may be produced by a Doctor A who desires certain rules to be followed when his patients are being treated on the medical device 120, for example being weaned from a ventilator. A second treatment protocol 211 may be produced by a Doctor B who desires a different set of rules to be followed for her patients when the patients are being weaned off the ventilator. In addition, a set of treatment protocols 212 may be produced by an organization such as a hospital or a national standards body such as a physicians association. As will be appreciated, numerous different treatment protocols may be produced by numerous parties and stored in database 215 as circumstances warrant.

Although shown as an external database, the database 215 need not be external to the protocol template generation and branching logic system 200 but can be integrated within the protocol template generation and branching logic system 200. The database 215 can be the treatment protocols 210, 211, and/or 212 themselves or it can be a compilation that is formatted in such a way as to provide the information in a recognizable form for retrieval. In some embodiments, the database 215 can be provided by the entity which has authored the treatment protocols. Additionally, in some embodiments, the database 215 can be automatically updated with the release of new treatment protocols.

In some embodiments, the treatment protocol retrieval module 205 receives a treatment protocol directly from a user 275, who may be a treatment provider. In such embodiments, the user 275 may use a User Interface (UI) 270 to directly enter the treatment protocol.

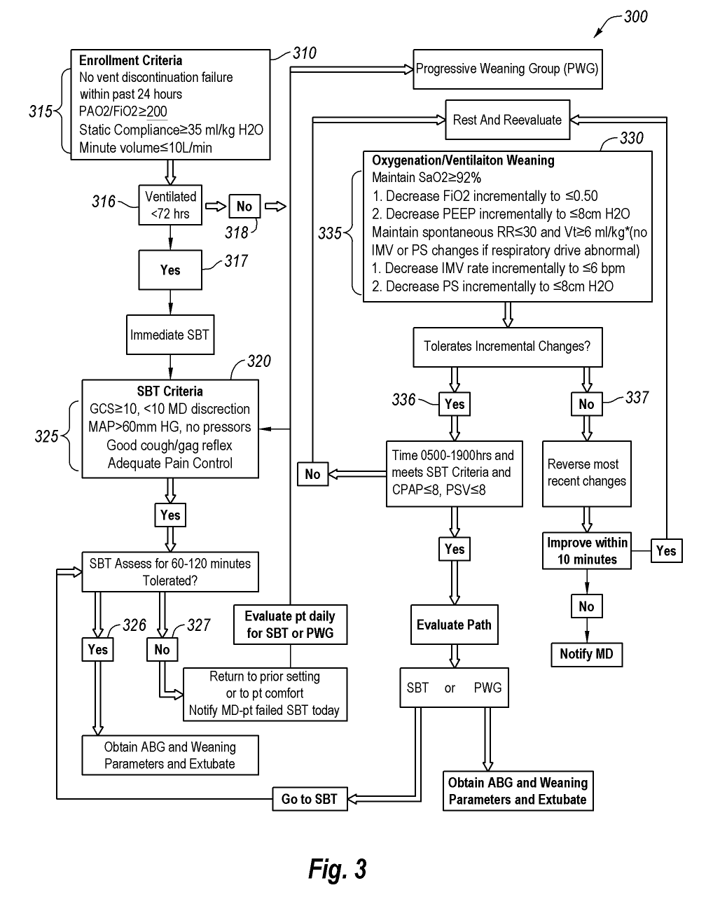

Attention is now given to FIG. 3, which illustrates an example treatment protocol 300 for weaning a patient off of a ventilator, which is an example of a medical device 120. As illustrated, the treatment protocol 300 includes various protocol steps or clinical decision points. For ease of illustration, only three of these protocol steps will be described although it will be appreciated that all of the steps or clinical decision points would be completed to successfully perform the treatment protocol 300. For example, a 310 includes rules 315 that specify if the treatment protocol step 310 is to be successfully performed or not. In addition, the treatment protocol step 315 includes a time interval 316 that specifies how much time the protocol step should be performed.

As mentioned previously, each treatment protocol step includes two clinical decision points or treatment pathways that followed depending on if the rules are successfully met or not. For example, as shown in FIG. 3, a first decision point 317 is followed if the rules 315 are successfully passed or met and the next step performed is the treatment protocol step 320. However, if the rules 315 are not passed or met, a second decision point 318 is followed and the next step is the treatment protocol step 330

A protocol step or clinical decision point 320 also indicates rules 325 that should be met in order for this protocol step or clinical decision point to be passed. If the rules 325 are passed or met, a first decision point 326 is followed to the next treatment protocol step. However, if the rules 325 are not passed or met, then a second decision point 327 is followed to the next treatment protocol step.

Likewise, a protocol step or clinical decision point 330 also indicates rules 335 that should be met in order for this protocol step or clinical decision point to be passed. If the rules 335 are passed or met, a first decision point 336 is followed to the next treatment protocol step. However, if the rules 335 are not passed or met, then a second decision point 337 is followed to the next treatment protocol step.

Returning to FIG. 2, the treatment retrieval module 205 presents the treatment protocols 210, 211 and/or 212 to a protocol template generation module 240 and/or to an alarm module 250. In some embodiments, the treatment protocols 210, 211 and/or 212 are presented to the protocol template generation module 240 in the same format or substantially the same format in which the treatment protocols 210, 211 and/or 212 were received from the database 215. In other embodiments, the format of the treatment protocols 210, 211 and/or 212 may be modified before the treatment protocols 210, 211 and/or 212 are presented to the protocol template generation module 240, the alarm module 250 or both.

The protocol template generation and branching logic system 200 also includes a medical device information retrieval module 220. As shown, the medical device module acts as an interface between the protocol template generation and branching logic system 200 and a medical device 225. It will be appreciated that the medical device 225 may be any reasonable medical device and may correspond to the medical device 120. In addition, it will be appreciated that the medical device 225 may represent an actual medical device that is connected to a patient and may also represent various other sub-systems that are connected between the actual medical device and the protocol template generation and branching logic system 200. As mentioned, the medical device 225 may be a ventilator in some embodiments.

In operation, the medical device 225 provides medical device information 226 to the medical device information retrieval module 220. The medical device information 226 may include information such as make and model that identify the medical device. The medical device information 226 may also include various operational parameters of the medical device. For example, in an embodiment where the medical device is a ventilator, the operational parameters may include whether the ventilator is plugged in, air flow, or is an air tube partially or fully blocked. In addition, the medical device information 226 may also include patient diagnostic information for a patient that is connected to the medical device. For example, in an embodiment where the medical device is a ventilator, the patient diagnostic information may include how many breaths per minute the patient is taking or how much air pressure is exerted on the lungs as the patient takes a breath. It will be appreciated that medical device information 226 may include numerous types of operational, patient diagnostic, or other types of medical device data as circumstances warrant.

The medical device information retrieval module 220 presents the medical device information 226 to the protocol template generation module 240 and/or to the alarm module 250. In some embodiments, the medical device information 226 is presented to the protocol template generation module 240 and to the alarm module 250 in the same format or substantially the same format in which the medical device information 226 was received from the medical device 225. In other embodiments, the format of the medical device information 226 may be modified before the medical device information 226 is presented to the protocol template generation module 240, the alarm module 250 or both.

The protocol template generation and branching logic system 200 further includes a patient information retrieval module 230. The patient information retrieval module acts as an interface between the protocol template generation and branching logic system 200 and a patient information database 235. In some embodiments, the patient information database 235 is a patient admission system for a hospital or other health care facility. The patient information database 235 is configured to hold patient information 236 about a patient that is connected to the medical device 225. The patient information 236 may include identification information such as name, age, who the patient's doctor is, and the like. In addition, the patient information 236 may include information that specifies the illness that has led to the patient being treated with the medical device 225. Other pertinent information that may be useful in treating the patient may also be included as part of the patient information 236. Although not shown in FIG. 2, the medical device 225 may also have access to the patient information database 235 so that the patient information may be coordinated with the actual patient connected to the medical device 225.

The patient information retrieval module 230 presents the patient information 236 to the protocol template generation module 240 and/or to the alarm module 250. In some embodiments, the patient information 236 is presented to the protocol template generation module 240 and to the alarm module 250 in the same format or substantially the same format in which the patient information 236 was received from the database 235. In other embodiments, the format of the patient information 236 may be modified before the patient information 236 is presented to the protocol template generation module 240, the alarm module 250 or both.

As previously mentioned, the treatment protocols 210, 211, and/or 212, the medical device information 226, and the patient information 236 are received by the protocol template generation module 240. In operation, the protocol template generation module 240 is configured to generate treatment protocol templates (also referred herein simply as treatment template or template) for patient treatment. The treatment protocol templates will typically include the various rules that should be met in order to satisfy the underlying treatment protocol 210, 211, or 212.

Using the accessed information or at least a portion of the information, the protocol template generation module 240 will generate a treatment protocol template for each step or clinical decision point in a treatment protocol. For instance, for the treatment protocol 210, the protocol template generation module 240 will generate a treatment protocol template 241A for a first step or clinical decision point in the treatment protocol, a treatment protocol template 241B for the second step or clinical decision point in the treatment protocol, and so on until every step or clinical decision point in the protocol 210 has a treatment protocol template generated for it. The ellipses illustrated by 241C are to show that since the treatment protocol 210 may have any number of steps or clinical decision points, there may be any number of corresponding treatment protocol templates 241 generated by the protocol template generation module 240. The treatment templates 241 may then be stored in a database 245 or some other database or memory that is accessible to the protocol template generation module 240.

Likewise, for the treatment protocol 211, the protocol template generation module 240 will generate a treatment protocol template 242A for a first step or clinical decision point in the treatment protocol, a treatment protocol template 242B for the second step or clinical decision point in the treatment protocol, and so on until every step or clinical decision point in the protocol 211 has a treatment protocol template generated for it. The ellipses illustrated by 242C are to show that since the treatment protocol 211 may have any number of steps or clinical decision points, there may be any number of corresponding treatment protocol templates 242 generated by the protocol template generation module 240. The treatment templates 242 may then be stored in the database 245 or some other database or memory that is accessible to the protocol template generation module 240.

In some embodiments, the treatment protocol templates may include or list various parameters that are associated with the rules of the treatment protocol step associated with a treatment protocol template. The parameters are used to test if the rules are passed or not passed during use. For example, a rule may specify a certain number of breaths per minute that a patient should produce. The template would include a breath parameter that would test or have inputted information on the actual number of breaths per minute. If the actual number met the rule, the parameter is passed. If the actual number does not meet the rule, the parameter is not passed.

For example, referring to again to the treatment protocol 300 illustrated FIG. 3, the protocol template generation module 240 will generate a treatment protocol template for the step or clinical decision point 310, another treatment protocol template for the step or clinical decision point 320, and a different treatment protocol template for the step or clinical decision point 330. As will be appreciated, the steps or clinical decision points of the treatment protocol 300 not specifically designated with a reference numeral will also have a treatment protocol template generated for them as well.

Returning to FIG. 2, in some embodiments, the templates 241 and 242 may be generated as web-based templates that are capable of being transmitted over the Internet or some other network. Accordingly, the protocol template generation and branching logic system 200 includes an interface 260 that is connected to the web-based UI 270. The web-based UI 270 may be an Internet browser running on a computer. Because the UI 270 and the templates 241and 242 may be web-based, they allow for the user 275 to be remotely located from the protocol template generation and branching logic system 200 and still have access to the system.

In those embodiments where the templates 241 and/or 242 are web-based templates, the templates are viewable on the web-based UI 270. The user 275, who may be a doctor or some other medical professional and may correspond to user 115, may access the templates 241 and/or 242. The user 275 may provide user input 276 in the form of checking boxes and the like to verify that treatment guidelines specified in the template have been completed or not.

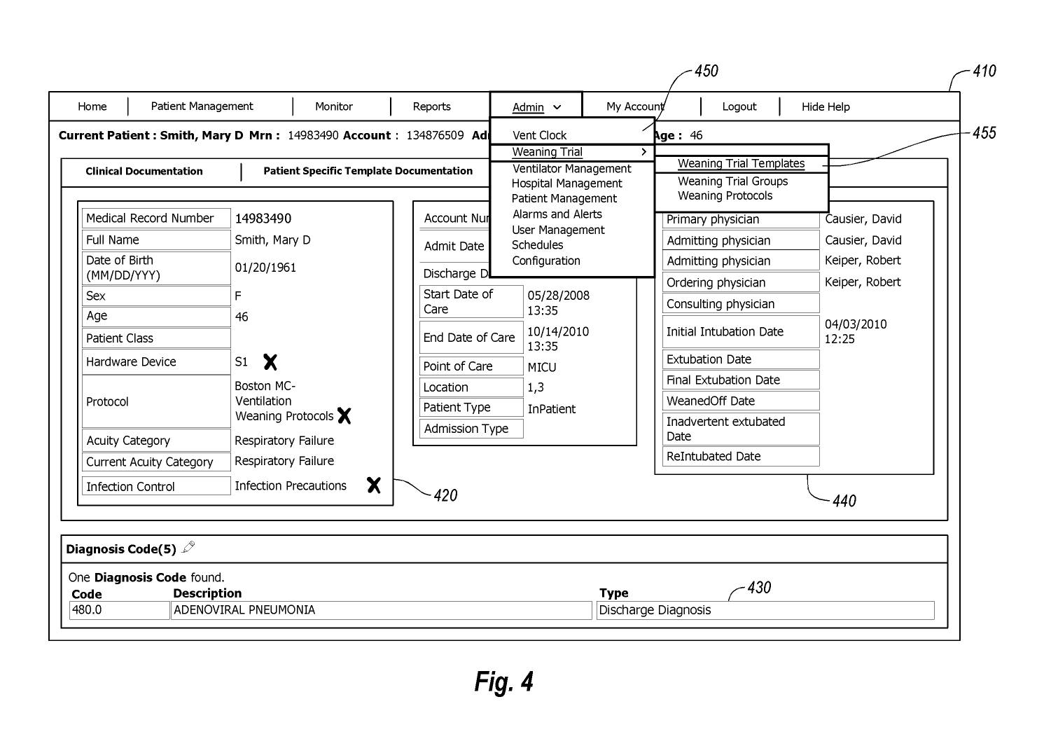

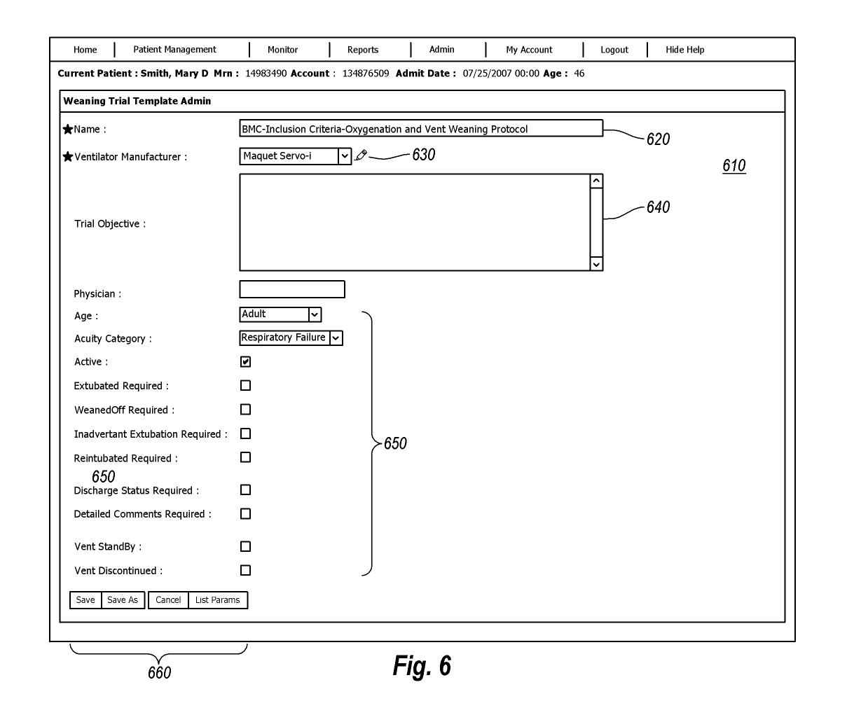

Attention is now given to FIGS. 4-8, which illustrate a specific embodiment of the protocol template generation and branching logic system 200, especially the protocol template generation module 240, generating web-based treatment protocol templates such as treatment templates 241 and 242 using the UI 270. As shown in FIG. 4, a user 275 accesses a first screen 410 that is used to help create a new treatment protocol template for a protocol that is used to wean a patient off of a ventilator. The screen 410 includes patient information 420 and a patient diagnosis 430 that is gathered from the patient database 235 as previously described. In addition, the screen 410 includes doctor and other information 440 that is gathered from the database 215.

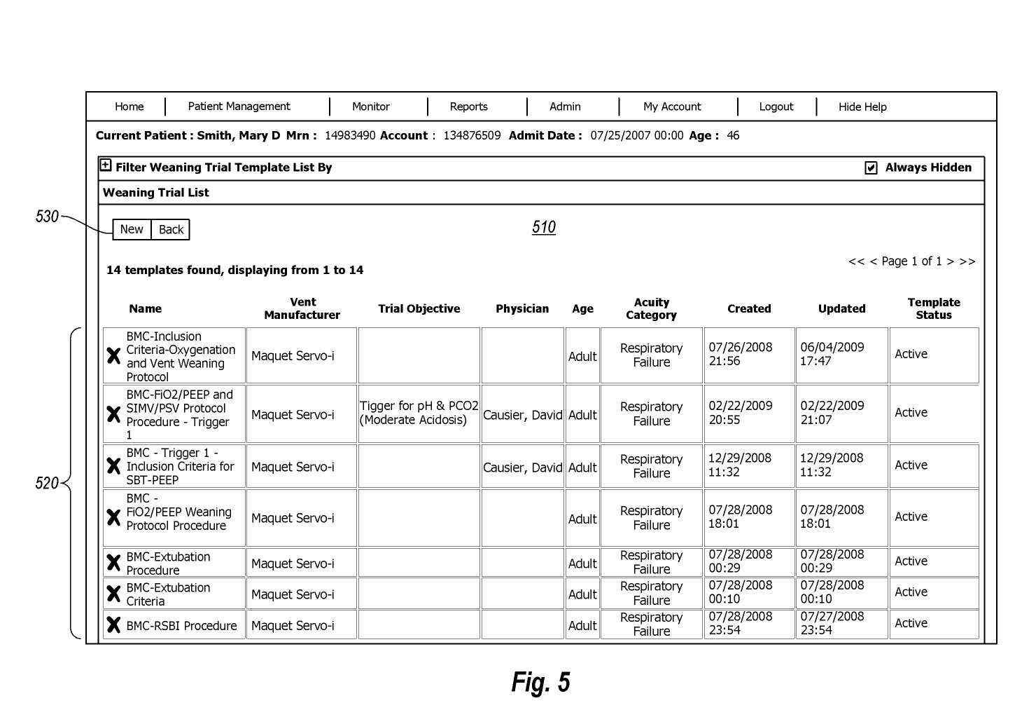

In operation, the user 275 selects an interface element 450 and an interface element 455 to access a weaning trial template screen. FIG. 5 illustrates a weaning trial templates screen 510, which lists various treatment templates 520 that have previously been generated. As illustrated, the weaning trial templates screen 510 shows a template name, ventilator information, trial objectives, physician information, target age group, category, creation and update dates, and status for each treatment template 520. The user 275 may select the interface element 530 in order to create a new treatment template.

Selecting the user interface element 530 opens a screen 610 that may be used to create the new treatment protocol template. The user 275 enters a name 620 for the treatment protocol template and ventilator information 630. The user 275 may also enter trial objective information 640 and other information 650 as needed. The user may then select one of the interface elements 660 to save the screen or to add new parameters to the template.

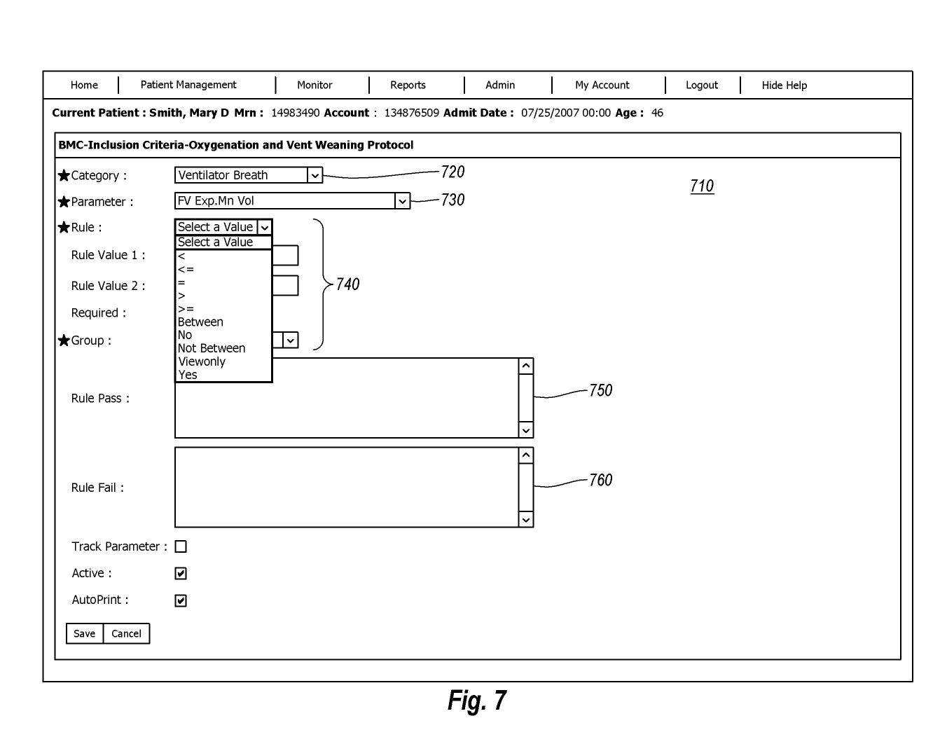

FIG. 7 illustrates a screen 710 that allows the user to select and configure a new parameter for the treatment protocol template. As shown, a category 720 and a parameter 730 are selected using their associated drop down lists. For the selected parameter, various rules 740 may then be selected. The rules 740 may specify a value, value range, or condition that must be met in order to stratify the parameter. For example, the rules may specify a yes/no value or some numerical number or numerical range that should be met. Additional descriptive Information regarding when a rule passes and when it fails may also be specified in boxes 750 and 760 respectively.

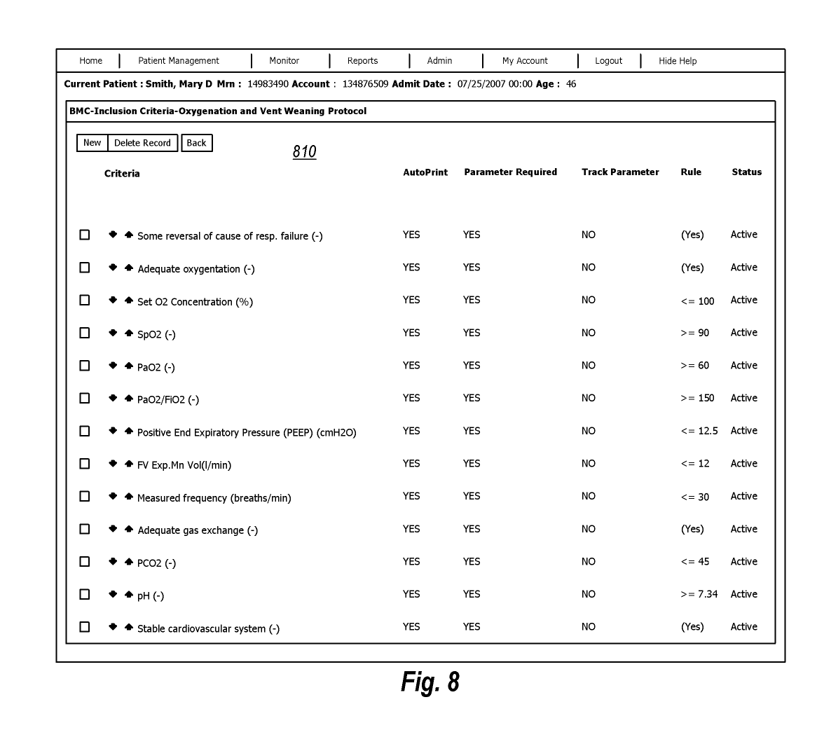

FIG. 8 illustrates the configuration screen 810 when all of the parameters have been added to the treatment protocol template. As shown, the screen 810 lists various parameters as parameter fields have an associated check-off box, their associated rule, and their current status. The user 275 may delete, modify, or move the position of the parameters as needed. Thus, when the steps illustrated in FIGS. 4-8 have been performed, a treatment protocol template will be created.

Attention is again made to FIG. 2, which further illustrates that the protocol template generation and branching logic system 200 includes a branching or next step logic module 280. The branching or next step logic module 280 is configured to access the generated treatment protocol templates 241 and/or 242 stored in database 235 or generated by the protocol template generation module 240. The branching or next step logic module 280 may then associate or link each treatment template with the treatment protocol step or clinical decision point in the treatment protocol that the treatment protocol template was created to cover. The next step logic module 280 is also configured to link or associate each treatment protocol step with the protocol treatment step that is to be performed next when the rules of the current treatment protocol step are passed and when the rules are not passed.

Further, the branching or next step logic module 280 is configured to determine if the various parameter fields and their associated rules of each treatment protocol template have been met or have not been met. If the rules associated with the parameter fields have been met, then the branching or next step logic module 280 specifies the next treatment protocol step that should be performed. Since the next treatment protocol step will have an associated treatment protocol template, the branching or next step logic module 280 will recommend that that template should be accessed. Likewise, if the rules associated with the parameter fields have not been met, then the branching or next step logic module 280 also specifies the next treatment protocol step and its associated template that should be accessed and followed. In some embodiments, the branching or next step logic module 280 may specify that the next treatment protocol step is in a different treatment protocol than the one currently being used. For example, a more intensive treatment protocol may be needed if the patient fails the original breathing tests. In such embodiments, the branching or next step logic module 280 will specify the new protocol and its associated templates to the user 275. Thus, the branching or next step logic module 280 is able to consistently determine and then specify what the next protocol step and associated protocol treatment template should be used during the treatment process. It will be appreciated that the branching or next step logic module 280 will continue to specify what the next protocol step and associated protocol treatment template should be until a treatment protocol is completed.

As will be appreciated, it will often be difficult for a user 275 to know what the next step in a treatment protocol should be. This is especially true in those circumstances when the rules associated with the parameter fields are not met. Advantageously, the branching or next step logic module 280 provides this information automatically to the user 275 in an easily understood manner.

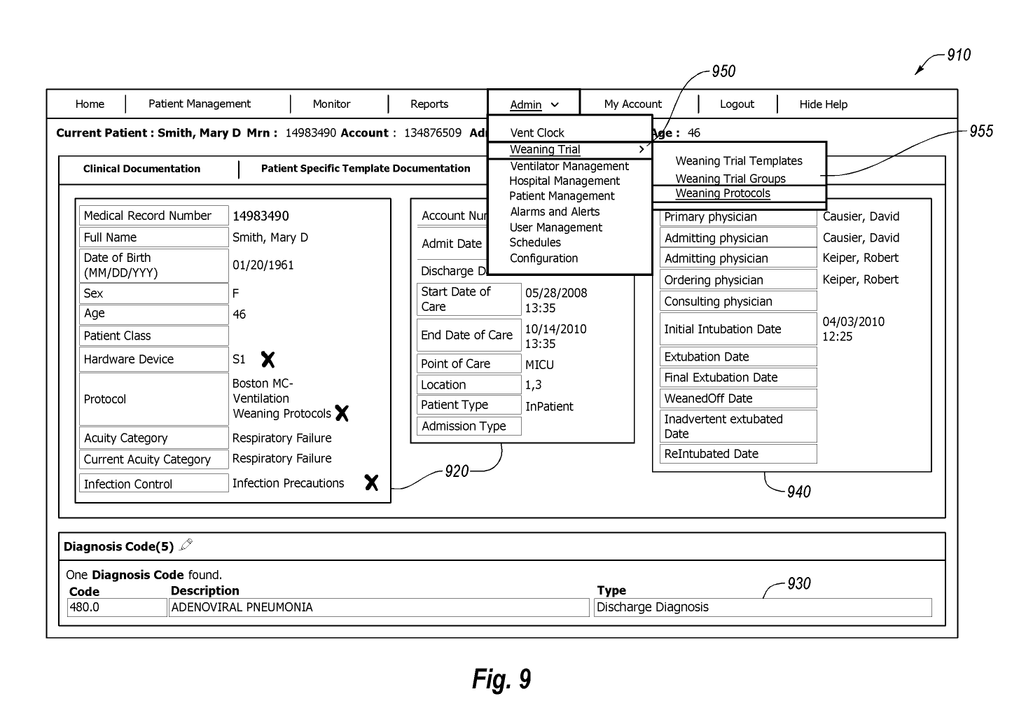

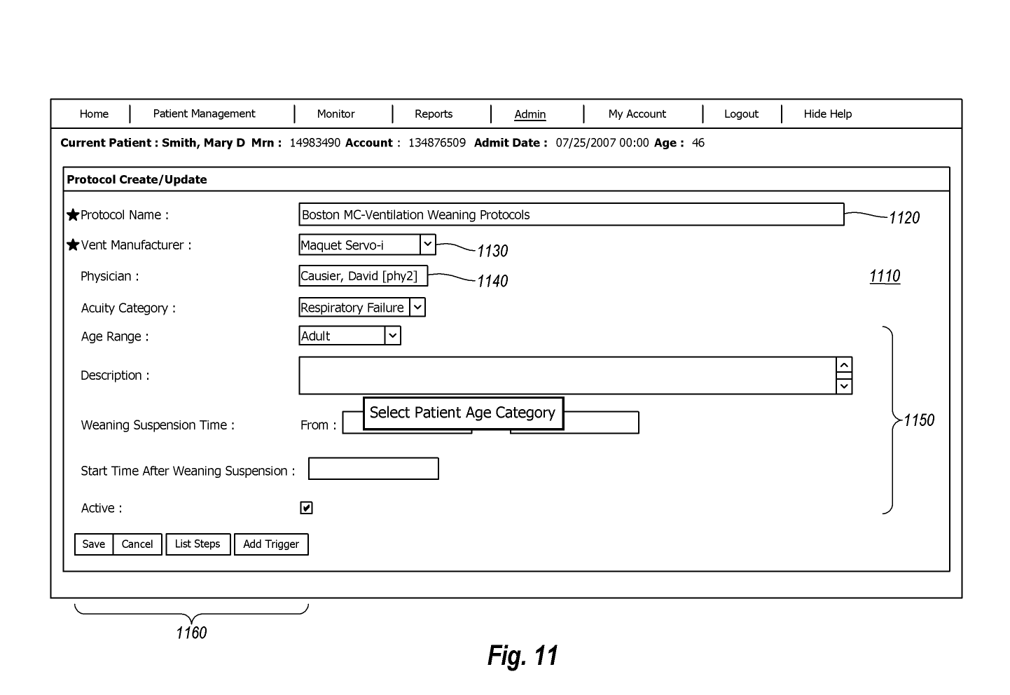

Attention is now given to FIGS. 9-13, which illustrate a specific embodiment of the protocol template generation and branching logic system 200, specifically the branching or next step logic module 280, associating treatment templates with a protocol and determining the next step in a treatment protocol pathway. As shown in FIG. 9, a user 275 accesses a screen 910 that is used to help select a ventilator weaning protocol. The screen 910 includes patient information 920 and a patient diagnosis 930 that is gathered from the patient database 235 as previously described. In addition, the screen 910 includes doctor and other information 940 that is gathered from the database 215.

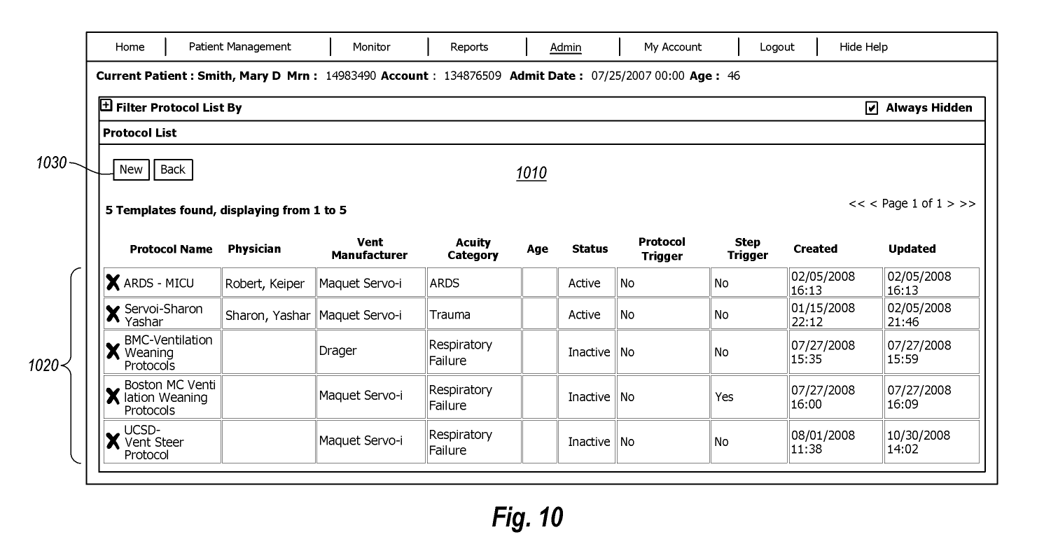

In operation, the user 275 selects an interface element 950 and an interface element 955 to access a weaning trial protocol or treatment pathway. FIG. 10 illustrates a protocol sheet 1010, which lists various treatment protocols 1020. As illustrated, the protocol screen 1010 shows a protocol name, ventilator information, protocol trigger, target age group, category, creation and update dates, and status for each treatment protocol 1020. The user 275 may select the interface element 1030 in order to create a new treatment template.

Selecting the user interface element 1030 opens a screen 1110 that may be used to create or update a treatment protocol. The user 275 enters a name 1120 for the treatment protocol and selects ventilator information 1130 using an interface element. The user 275 may also enter doctor information 1140 and may enter or select other information 1150 as needed. The user may then select one of the interface elements 1160 to save the screen or to add new steps to the protocol or to link to other protocols.

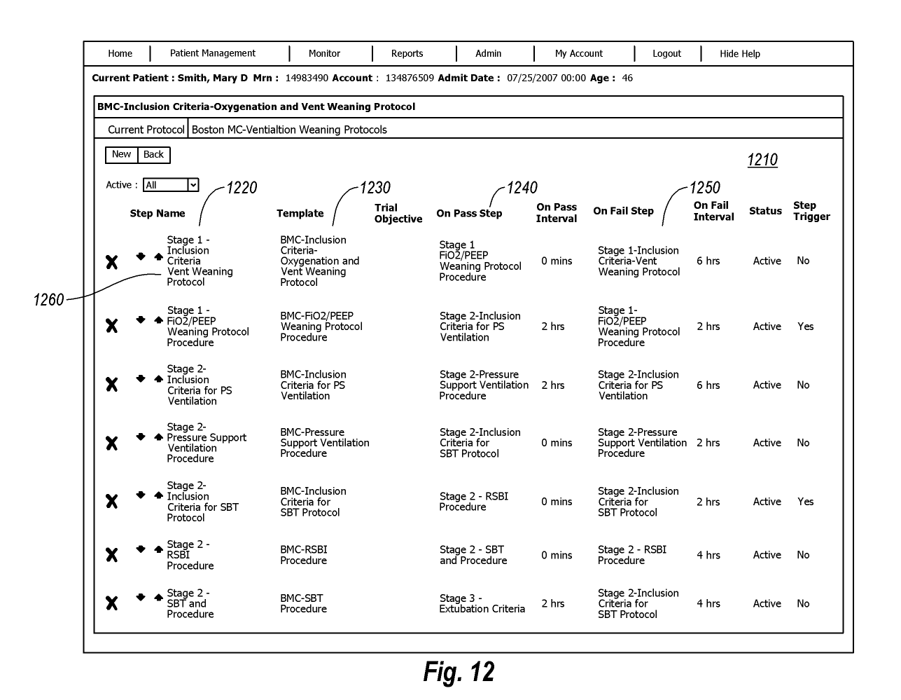

FIG. 12 illustrates a screen 1210 that lists the various steps of a protocol selected in screen 1110 and the treatment templates associated with each step of the protocol. For example, a first step 1220 has a template 1230 associated with it. Further, the step 1220 indicates at 1240 the next step to follow if the parameters and rules of the treatment template 1230 are successfully performed. In this case, the next step is the next step after 1260 in the current protocol. The step 1220 also indicates at 1250 the next step to follow if the parameters and rules of the treatment template 1230 are not successfully performed. In this case, the next step is to repeat the protocol step 1220 in six hours.

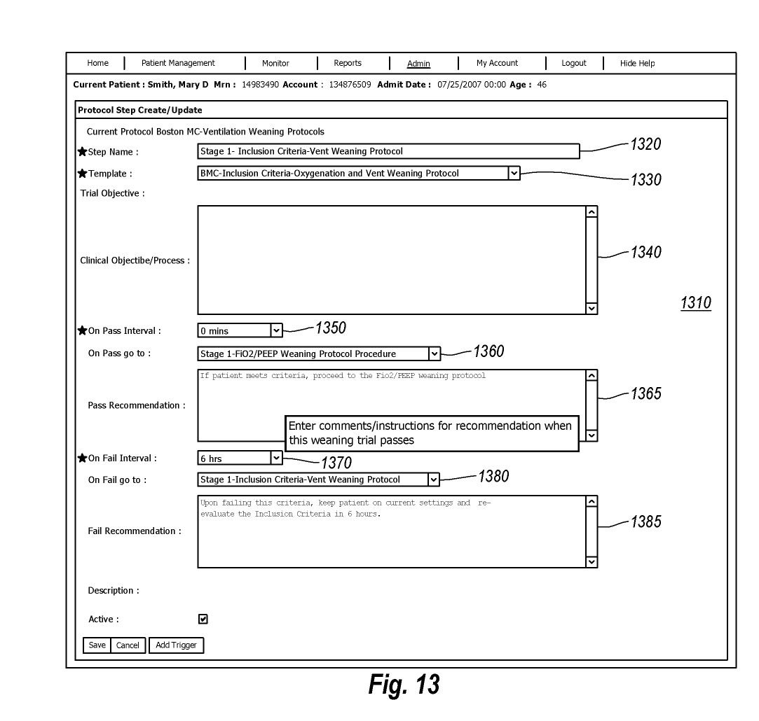

FIG. 13 illustrates a screen 1310 for associating a treatment protocol step with a treatment template and for configuring the next treatment protocol step that will be followed after the current protocol step. For example, a treatment protocol step name may be entered into box 1320. A treatment protocol template for association with the treatment protocol step may be selected by a drop down list of treatment protocol templates previously generated as previously discussed as shown at 1330. Clinical objectives may be entered at box 1340. A drop down list allows for the selection of a pass time interval at 1350.

At 1360, a drop down list allows for the selection of the next treatment protocol step in the current protocol that will be accessed if the rules associated with the current treatment protocol step are successfully passed or met. The treatment protocol step designated at 1360 is the “On-Pass” treatment protocol step previously discussed and will typically have a treatment protocol template associated with it that includes parameter fields and associated rules. Thus, the treatment template of the current treatment protocol step is linked to the template that is associated with the next or “On-Pass” treatment protocol step. A box 1365 allows for the input of a recommendation to follow when the current treatment protocol step is passed. This recommendation will generally correspond to the treatment protocol step designated at 1360.

At 1370, a drop down list allows for the selection of a time interval if the current treatment protocol step is not successfully performed. At 1380, a drop down list allows for the selection of the next treatment protocol step in the current protocol or a treatment protocol step in another protocol that will be accessed if the rules associated with the current treatment protocol step are not successfully passed or met. The treatment protocol step designated at 1380 is the “On-Fail” treatment protocol step previously discussed and will typically have a treatment protocol template associated with it that includes parameter fields and associated rules. Thus, the treatment template of the current treatment protocol step is linked to the template that is associated with the next or “On-Fail” treatment protocol step. A box 1385 allows for the input of a recommendation to follow when the current step treatment protocol fails. This recommendation will generally correspond to the treatment protocol step designated at 1380.

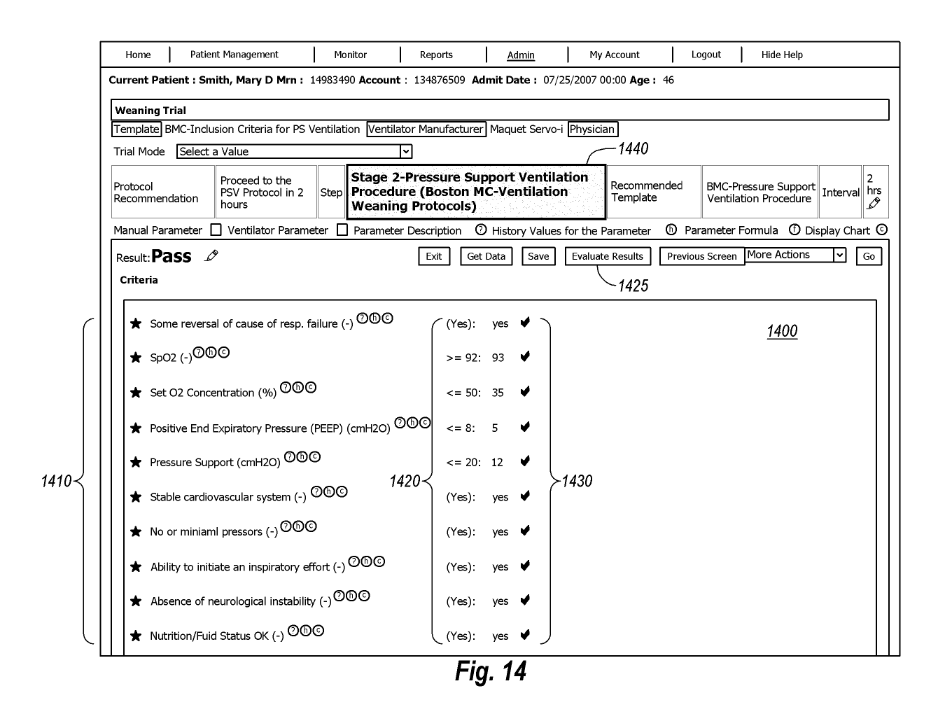

Attention is now made to FIG. 14, which shows an example of a treatment protocol template 241 or 242, designated at 1400 that has been generated and associated with a treatment protocol step in the manner previously described and that is used during a clinical procedure when a patient is hooked up to an actual ventilator. As shown, the template 1400 includes treatment parameter fields 1410 that specify various rules that must be satisfied for the treatment protocol step to be successfully performed. The treatment protocol template 1400 also includes actual values 1420 that represent the actual ventilator information 226 that is read from the ventilator connected to the patient and other actual diagnostic and clinical information regarding the patient which may be obtained from other sources or that may be entered by the clinician. That is, some of the actual values 1420 are diagnostic information automatically entered electronically into the parameter fields 1410 from the ventilator. Other actual values 1420 are manually inputted into the parameter fields 1410 by a treatment provider based on patient observation or measured data. Additional actual values 1420 may be received from sources outside of the protocol template generation and branching logic system 200. The user 275 may click the evaluate interface element 1425 to validate that the actual values 1420 are within the limits specified by the underlying rules associated with parameter fields 1410 that have been selected as described. As shown, the check boxes 1430 indicate that the rules have all been passed or met.

As mentioned, the branching or next step logic module 280 is configured to determine if the rules associated with the parameter fields 1410 were met by the actual values 1420. If they are met, then the branching or next step logic module 280 determines the next treatment protocol step that should be performed. Likewise, if the rules associated with the parameter fields 1410 were not met, then the branching or next step logic module 280 determines the next treatment protocol step that should be performed. In some embodiments, a new treatment protocol based on the factors such as ventilator model and attending doctor may be selected. For example, a more intensive treatment protocol may be needed if the patient fails the original breathing tests.

Regardless of pass or fail, the branching or next step logic module 280 provides a recommendation for the next treatment protocol step and its associated template at the time the user 275 selects the evaluate results button 1425. This is shown in FIG. 14 at 1440. The user 275 may then select this recommended treatment protocol step and its associated template, which will then be displayed to the user on UI 270.

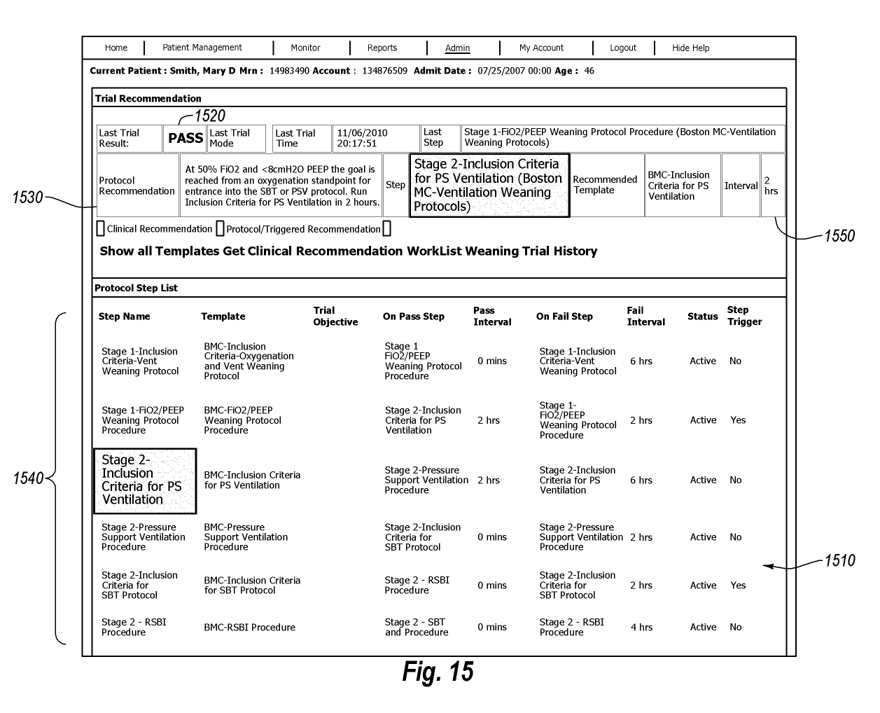

The user 275 may also access a screen 1510 shown in FIG. 15. This screen shows the last treatment protocol step performed and if was successful as seen at 1520. In addition, this screen shows the recommended next treatment protocol step at 1530, which can be selected by the user 275. Further, a list 1540 of the current treatment protocol steps is also shown. This screen may be advantageous to keep track of what the next treatment protocol step should be when an interval 1550, which in this case is two hours, should pass before the next treatment protocol step is performed.

It will be appreciated that the branching or next step logic module 280 provides significant advantages. For example, the user 275 is not required to determine what the next step in the treatment protocol should be as this is done automatically for him or her. This removes the need for the user 275 to have to manually follow complex treatment protocols.

Returning to FIG. 2, the protocol template generation and branching logic system 200 includes a report module 290. In operation, the report module 290 is configured to track and record every action taken by the user 275 when utilizing the templates 241. In this way, it can be easily determined if the user 275 complied with the treatment protocol steps specified in the template 241. In addition, in those situations where the user 275 determines that a deviation from the treatment protocol is needed, the report module 290 will record this and allow the user 275 to specify the reasons why the user felt the deviation was needed. Further, the report module 290 will record those situations where the user 275 deviates from the treatment protocols for unexplained reasons.

In some embodiments, the report module 290 may be used to measure the effectiveness of various treatment protocols 210. As discussed previously, it is often the case that Doctor A and Doctor B will specify different treatment protocols 210 for similarly situated patients who are connected to a given type of medical device 120. As will be appreciated, it may be the case that the treatment protocols specified by Doctor A are more effective than treatment protocols specified by Doctor B. Such information would be useful to the doctors and the hospital. However, conventionally there has been no easy way to compare the guidelines or to determine which one was more effective. Further, even if such comparison data were available, there has been no easy way to communicate this data to the doctors and the hospital.

Advantageously, the report module 290 is able to track the effectiveness of each doctor's treatment protocols by measuring such metrics as the time it takes and the number of steps needed to effectively treat the patient using the medical device 120. Over time, the report module can determine which treatment protocols are more effective. This information may then be provided to the doctors and the hospital to help in generating the most effective treatment protocols.

This data on effective treatment protocols may be gathered across a wide variety of hospitals, health organizations and the like. The data can then be compared and benchmarks for each one determined and compared to each other. In this way, interested parties will be able to determine what treatment protocols appear to be most effective and what changes they can make to their own treatment protocols.

In some embodiments, the report module 290 may also be configured to generate graphs and tables regarding the results of a given treatment protocol. Such data may be helpful to the user 275 when treating the patient.

Attention is again to protocol template generation module 240. In addition to generating treatment protocol templates 241 and 242 as previously discussed, the protocol template generation module 240 is also able to generate a template 243 for electronic medical device 120 checks.

In operation, the protocol template generation module 240 accesses the medical device information 226 and uses this information to generate the web-based template 243. In addition, the protocol template generation module 240 accesses device rules, which may be part of information 226, that specify desirable operating parameters. This template may then be provided to UI 270 as described to allow the user 275 access to the template. As with the templates 241 or 242, the template 243 may be viewed remotely by the user 275 over the Internet or some other network as it is web-based.

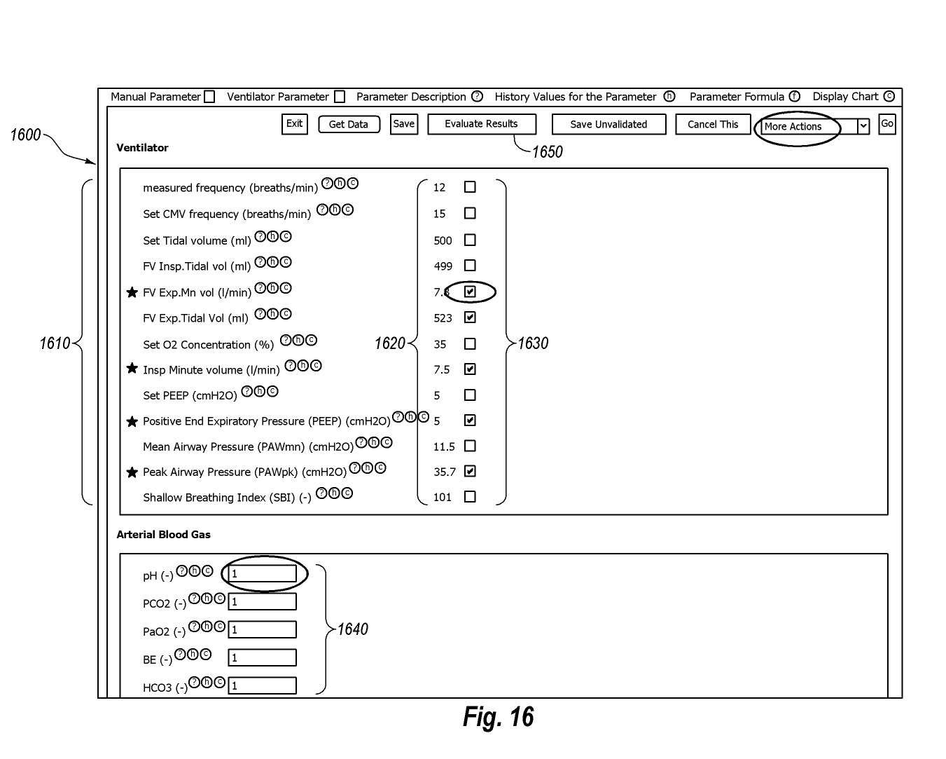

Turning to FIG. 16, an example of a template 243 for a medical device 120 implemented as a ventilator, designated at 1600, is shown. Template 1600 includes ventilator operating parameters 1610 and actual values 1620 that have been read from ventilator 225.

In addition, the template 1600 includes checkboxes 1630 and manual data boxes 1640. In order to validate the ventilator, the user 275 will check the required boxes 1630 and enter any required data into boxes 1640. The user 275 then may select the evaluate results 1650 button. If the actual values 1620 and any manually entered data are within acceptable ranges, the protocol template generation and branching logic system 200 will validate the ventilator 225.

As with the embodiments for treatment protocols, the report module 290 is configured to generate graphs and tables showing trending data for the ventilator 225. In addition, the report module 290 is able to record that verification took place as prescribed and if not, the reasons why not.

It will be appreciated that the protocol template generation module 240 allows for customization of both the templates 241, 242 and 243. That is, the user 275 or some other person or system is able to specify data fields as desired. These fields will then be automatically added by protocol template generation module 240 to the templates 241, 242 and/or 243. Further, such fields can be automatically removed or rearranged by protocol template generation module 240 as circumstances warrant.

Returning again to FIG. 2, it is shown that protocol template generation and branching logic system 200 also includes an alarm module 250 including a waveform generator 255. In operation, the alarm module 250 is configured to receive the various input data as described above. The alarm module 250 may then generate audio and visual alarms based on this input. For example, if an operational parameter or a patient diagnostic parameter falls below a predetermined acceptable level, the alarm module 250 may generate the alarm. As with the templates, the alarms may be sent in real time to a user 275 that is remote from the protocol template generation and branching logic system 200. The alarm module 250 is further configured to allow the user 275 to prioritize the alarms based on importance. That is, an alarm for one parameter that is more critical than another may be configured to be generated before the other alarm.

As mentioned, the alarm module 250 may include a wave form generator 255. The waveform generator is configured to generate a real time waveform that looks at the mechanics of a diagnostic parameter such as a patient's lung pressure or heart beat. For example, in the case of a ventilator, when the patient takes a breath while connected to the ventilator, the pressure it took to push air into the lungs may be shown as a pressure curve. In addition, data regarding the volume of air in the lung at a given point of time may also be shown as a curve. Thus, the waveform generator produces the real time web-based waveforms and other visual graphs that allow the user 275 in real time to evaluate the health of the lung and treat it accordingly. If the waveforms are below acceptable levels, then alarms may be generated. It will be appreciated that any number different waveforms for various measurable data may be generated by waveform generator 255.

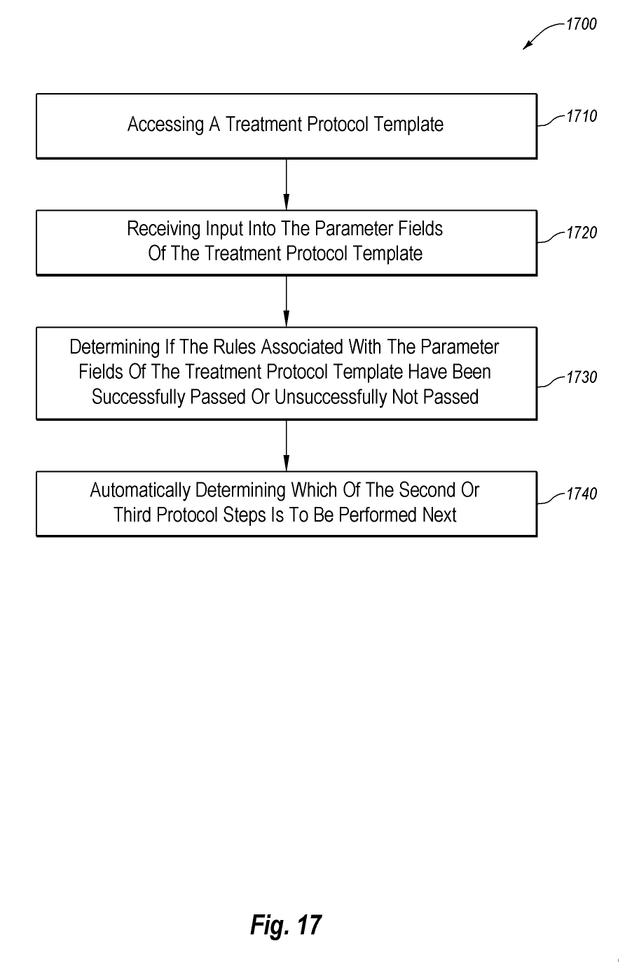

Attention is now given to FIG. 17, which illustrates a flow chart of a method 1700 for determining a next step that is to be performed in a patient treatment protocol upon completion of a current treatment protocol step. The method 1700 includes accessing 1710 a treatment protocol template. The treatment protocol template may be associated with a treatment protocol step of an underlying treatment protocol. The treatment protocol step includes rules that determine when the treatment protocol step is passed or not passed. The treatment protocol step includes a first clinical decision point that specifies a second protocol step that is be performed next when the rules are passed. The treatment protocol step also includes a second clinical decision point that specifies a third protocol step that is to be performed next when the rules are not passed. The treatment protocol template includes one or more parameter fields that are associated with at least one of the rules of the treatment protocol step. For example, the treatment protocol template 241A may be accessed by the protocol template generation and branching logic system 200. The treatment protocol template 241A may include parameter fields such as those seen in FIG. 14 discussed previously.

As previously described, the treatment protocol template 241A may be associated with a treatment protocol step, such as protocol step 310 of FIG. 3. As previously discussed, the protocol step 315 includes rules 315 that determine or specify when the protocol step passed or not passed. In other words, the rules 315 specify the criteria or conditions that should be complied with if the protocol step 310 is to be passed. If the criteria or conditions are not complied with, the protocol step 310 is not passed.

As also previously described, the protocol step 310 includes a first clinical decision point 317 that specifies the next protocol step to follow if the rules 315 are passed. The protocol step 310 also includes a second clinical decision point 318 that specifies the next protocol step to follow if the rules 315 are not passed.

The method 1700 includes receiving 1720 input into the parameter fields of the treatment protocol template. For example, the protocol template generation and branching logic system 200 may receive input into the parameter fields of the treatment protocol template in the manner previously described in relation to FIG. 14. As discussed, the input may be diagnostic information related to the medical device 225 or clinical information related to the patient. The information may be automatically input or may be manually input.

The method 1700 further includes determining 1730, based on the input, if the rules associated with the parameter fields of the treatment protocol template have been successfully passed or unsuccessfully not passed. For example, the protocol template generation and branching logic system 200 may determine, based in the information, if rules associated with the parameter fields of the treatment protocol template 241A have been successfully passed or unsuccessfully not passed.

The method 1700 further includes, in response to determining if the rules associated with the parameter fields have passed or not passed, automatically determining 1740 which of the second or third protocol steps is to be performed next For example, the protocol template generation and branching logic system 200 may determine which treatment protocol step, for example treatment protocol step 320 or treatment protocol step 330, to perform next in the manner previously described.

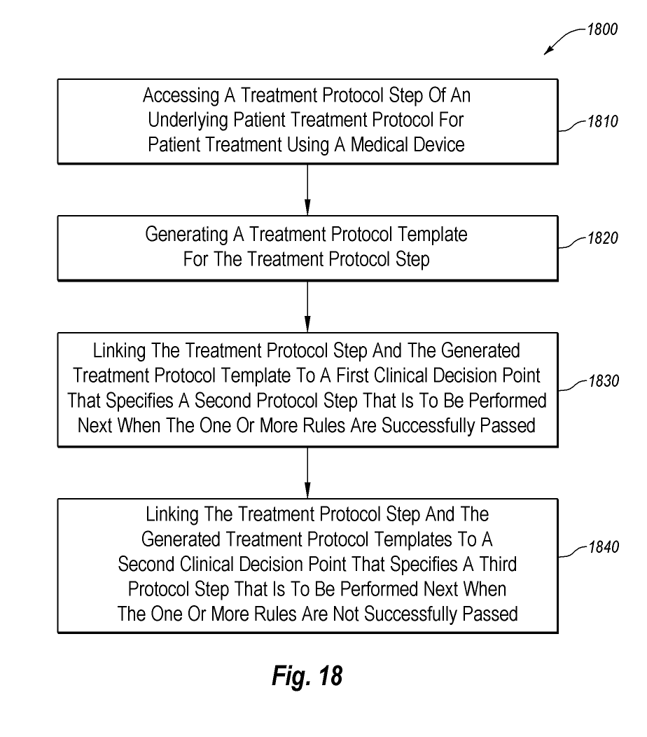

FIG. 18 illustrates a method 1800 for linking a next treatment protocol step with a protocol treatment template of a current treatment protocol step. The method 1800 includes accessing 1810 a treatment protocol step of an underlying patient treatment protocol for patient treatment using a medical device. The treatment protocol step includes one or more rules that are to be complied with in order to successfully perform the treatment protocol step. For example, the protocol template generation and branching logic system 200 may access a treatment protocol step such as treatment protocol step 1320.

The method 1800 also includes generating 1820 a treatment protocol template for the treatment protocol step. The treatment protocol template includes parameter fields associated with the one or more rules of the treatment protocol step. For example, the protocol template generation and branching logic system 200, especially the protocol template generation module 240, may generate a treatment protocol template 241A that includes the parameter fields as discussed.

The method 1800 further includes linking 1830 the treatment protocol step and the generated treatment protocol template to a first clinical decision point that specifies a second protocol step that is to be performed next when the one or more rules are successfully passed. For example, the protocol template generation and branching logic system 200 may link the treatment protocol step to a first clinical decision point. For instance, as illustrated in FIG. 13, the clinical decision point or “On-Pass” treatment protocol step 1360 may be linked to the treatment protocol step 1320. When the rules of the treatment protocol step 1320 are passed, the treatment protocol step 1360 is automatically accessed and may then be followed.

The method 1800 also includes linking 1840 the treatment protocol step and the generated treatment protocol template to a second clinical decision point that specifies a third protocol step is to be performed next when the one or more rules are not successfully passed. For example, the protocol template generation and branching logic system 200 may link the treatment protocol step to a second clinical decision point. For instance, as illustrated in FIG. 13, the clinical decision point or “On-Fail” treatment protocol step 1380 may be linked to the treatment protocol step 1320. When the rules of the treatment protocol step 1320 are not passed, the treatment protocol step 1380 is automatically accessed and may then be followed.

Embodiments of the present invention may comprise or utilize a special purpose or general-purpose computer including computer hardware, such as, for example, one or more processors and system memory, as discussed in greater detail below. Embodiments within the scope of the present invention also include physical and other computer-readable media for carrying or storing computer-executable instructions and/or data structures. Such computer-readable media can be any available media that can be accessed by a general purpose or special purpose computer system. Computer-readable media that store computer-executable instructions are computer storage media (devices). Computer-readable media that carry computer-executable instructions are transmission media. Thus, by way of example, and not limitation, embodiments of the invention can comprise at least two distinctly different kinds of computer-readable media: non-transitory computer storage media (devices) and transmission media.

Computer storage media (devices) includes RAM, ROM, EEPROM, CD-ROM or other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry or desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to computer storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that computer storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, and the like. The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices. The invention may also be practiced in a cloud computing environment using standard cloud computing resources.

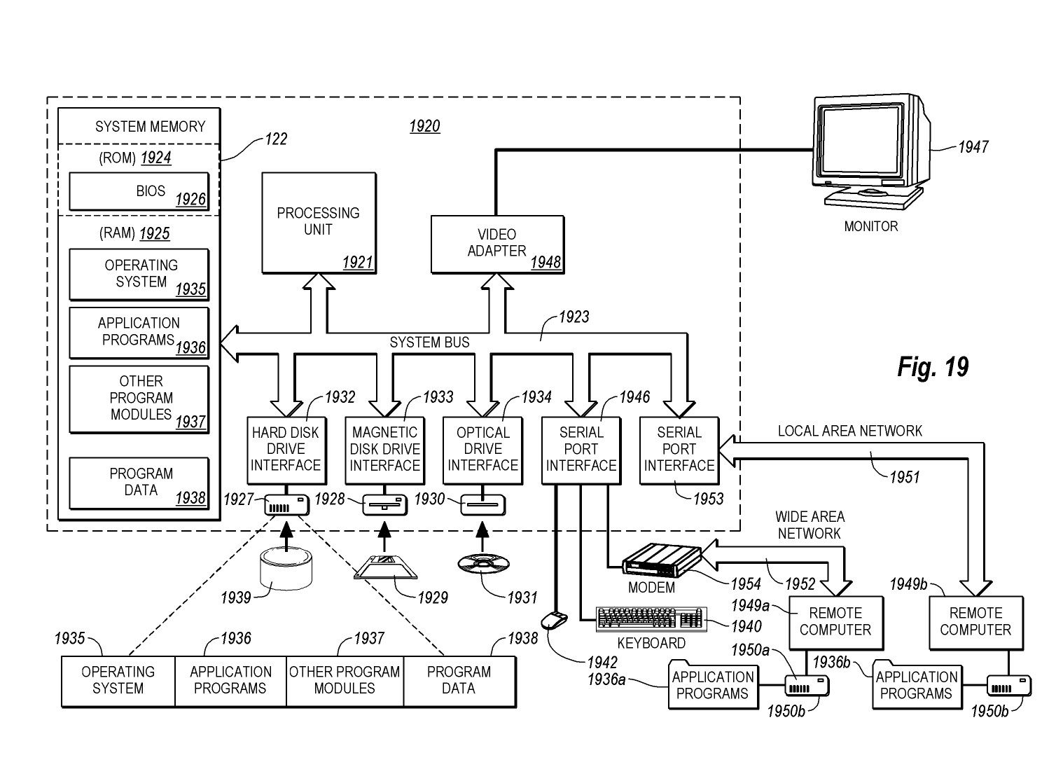

FIG. 19 and the following discussion are intended to provide a brief, general description of a suitable computing environment in which the invention may be implemented. Although not required, the invention will be described in the general context of computer-executable instructions, such as program modules, being executed by computers in network environments. Generally, program modules include routines, programs, objects, components, data structures, etc. that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

With reference to FIG. 19, an example system for implementing the invention includes a general purpose computing device in the form of a conventional computer 1920, including a processing unit 1921, a system memory 1922, and a system bus 1923 that couples various system components including the system memory 1922 to the processing unit 1921. It should be noted however, that as mobile phones become more sophisticated, they are beginning to incorporate many of the components illustrated for conventional computer 1920. Accordingly, with relatively minor adjustments, mostly with respect to input/output devices, the description of conventional computer 1920 applies equally to mobile phones. The system bus 1923 may be any of several types of bus structures including a memory bus or memory controller, a peripheral bus, and a local bus using any of a variety of bus architectures. The system memory includes read only memory (ROM) 1924 and random access memory (RAM) 1925. A basic input/output system (BIOS) 1926, containing the basic routines that help transfer information between elements within the computer 1920, such as during start-up, may be stored in ROM 1924.

The computer 1920 may also include a magnetic hard disk drive 1927 for reading from and writing to a magnetic hard disk 1939, a magnetic disk drive 1928 for reading from or writing to a removable magnetic disk 1929, and an optical disc drive 30 for reading from or writing to removable optical disc 1931 such as a CD-ROM or other optical media. The magnetic hard disk drive 1927, magnetic disk drive 1928, and optical disc drive 1930 are connected to the system bus 1923 by a hard disk drive interface 1932, a magnetic disk drive-interface 1933, and an optical drive interface 1934, respectively. The drives and their associated computer-readable media provide nonvolatile storage of computer-executable instructions, data structures, program modules and other data for the computer 1920. Although the exemplary environment described herein employs a magnetic hard disk 1939, a removable magnetic disk 1929 and a removable optical disc 1931, other types of computer readable media for storing data can be used, including magnetic cassettes, flash memory cards, digital versatile discs, Bernoulli cartridges, RAMs, ROMs, and the like.

Program code means comprising one or more program modules may be stored on the hard disk 1939, magnetic disk 1929, optical disc 1931, ROM 1924 or RAM 1925, including an operating system 1935, one or more application programs 1936, other program modules 1937, and program data 1938. A user may enter commands and information into the computer 1920 through keyboard 1940, pointing device 1942, or other input devices (not shown), such as a microphone, joy stick, game pad, satellite dish, scanner, or the like. These and other input devices are often connected to the processing unit 1921 through a serial port interface 1946 coupled to system bus 1923. Alternatively, the input devices may be connected by other interfaces, such as a parallel port, a game port or a universal serial bus (USB). A monitor 1947 or another display device is also connected to system bus 1923 via an interface, such as video adapter 1948. In addition to the monitor, personal computers typically include other peripheral output devices (not shown), such as speakers and printers.

The computer 1920 may operate in a networked environment using logical connections to one or more remote computers, such as remote computers 1949a and 1949b. Remote computers 1949a and 1949b may each be another personal computer, a server, a router, a network PC, a peer device or other common network node, and typically include many or all of the elements described above relative to the computer 1920, although only memory storage devices 1950a and 1950b and their associated application programs 1936a and 1936b have been illustrated in FIG. 19. The logical connections depicted in FIG. 19 include a local area network (LAN) 1951 and a wide area network (WAN) 1952 that are presented here by way of example and not limitation. Such networking environments are commonplace in office-wide or enterprise-wide computer networks, intranets and the Internet.

When used in a LAN networking environment, the computer 1920 is connected to the local network 1951 through a network interface or adapter 1953. When used in a WAN networking environment, the computer 1920 may include a modem 1954, a wireless link, or other means for establishing communications over the wide area network 1952, such as the Internet. The modem 1954, which may be internal or external, is connected to the system bus 1923 via the serial port interface 1946. In a networked environment, program modules depicted relative to the computer 1920, or portions thereof, may be stored in the remote memory storage device. It will be appreciated that the network connections shown are exemplary and other means of establishing communications over wide area network 1952 may be used.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for determining a next protocol step that is to be performed in a patient treatment protocol upon completion of a current treatment protocol step, the method comprising:
accessing a treatment protocol template, the treatment protocol template being associated with a treatment protocol step of an underlying patient treatment protocol, the treatment protocol step including rules that determine when the protocol step is passed or not passed, the treatment protocol step including a first clinical decision point that specifies a second protocol step that is be performed next when the rules are passed and including a second clinical decision point that specifies a third protocol step that is to be performed next when the rules are not passed, the treatment protocol template including one or more parameter fields that are associated with at least one of the rules of the treatment protocol step;
receiving input into the parameter fields of the treatment protocol template;
determining, based on the received input, if the rules associated with the parameter fields of the treatment protocol template have been successfully passed or unsuccessfully not passed; and
in response to determining if the rules associated with the parameter fields have passed or not passed, automatically determining which of the second or third protocol steps is to be performed next.

2. The method of claim 1, further comprising:
accessing a second treatment protocol template associated with the second treatment protocol step, the second treatment protocol step including rules that determine when the second protocol step is passed or failed, the second treatment protocol step specifying a fourth protocol step that is be performed when the second rules are passed and a fifth protocol step that is to be performed when the rules are not passed, the second treatment protocol template including one or more parameter fields that are associated with at least one of the rules of the second treatment protocol step;
receiving input into the parameter fields of the second treatment protocol template;
determining, based on the received input, if the rules associated with the parameter fields of the second treatment protocol template have been successfully passed or unsuccessfully not passed; and
in response to determining if the rules associated with the parameter fields have passed or not passed, automatically determining which of the fourth or fifth protocol steps is to be performed next.

3. The method of claim 1, wherein the input received into the parameter fields is at least one of clinical patient information related to a patient connected to a medical device that is associated with the underlying treatment protocol, information related to the medical device, other clinical or diagnostic information related to the underlying treatment protocol, wherein the input is received automatically into the parameter fields or is input manually into the parameter fields.

4. The method of claim 3, wherein the medical device is a ventilator and the underlying treatment protocol is a ventilator weaning protocol.

5. The method of claim 1, wherein the parameter fields of the treatment protocol template includes one or more user input blocks that are configured to manually receive user input that is indicative of clinical and diagnostic information related to the underlying treatment protocol.

6. The method of claim 1, wherein the third treatment protocol step is the same as the treatment protocol step such that the treatment protocol step and its associated treatment protocol templates are repeated when the rules are not passed.

7. Computer readable storage media having stored thereon computer readable instructions that when executed by a processor, cause a computing system to perform the method of claim 1.

8. A method linking a next treatment protocol step with a protocol treatment template of a current treatment protocol step the method comprising:
accessing a treatment protocol step of an underlying patient treatment protocol for patient treatment using a medical device, the treatment protocol step including one or more rules that are to be complied with in order to successfully perform the treatment protocol step;
generating a treatment protocol template for the treatment protocol step, the treatment protocol template including parameter fields associated with the one or more rules of the treatment protocol step;
linking the treatment protocol step and the generated treatment protocol template to a first clinical decision point that specifies a second protocol step that is to be performed next when the one or more rules are successfully passed and
linking the treatment protocol step and the generated treatment protocol template to a second clinical decision point that specifies a third protocol step that is to be performed next when the one or more rules are not successfully passed.

9. The method of claim 8, further comprising:
associating a first time interval with the generated treatment protocol template that indicates the time amount that should pass before the second treatment protocol step is accessed when the rules associated with the parameter fields of the generated treatment protocol templates are successfully passed; and
associating a second time interval with the generated treatment protocol template that indicates the time amount that should pass before the third treatment protocol step is accessed when the rules associated with the parameter fields of the generated treatment protocol templates are not successfully passed.

10. The method of claim 8, wherein the medical device is a ventilator and the treatment protocol is a ventilator weaning protocol.

11. The method of claim 8, further comprising:
accessing generated treatment protocol template;
receiving diagnostic information related to the medical device;
receiving clinical patient information related to a patient connected to the medical device; and
based on the received diagnostic information and clinical patient information, determining which of the second or third treatment protocol steps and their respective protocol treatment templates are to accessed next.

12. The method in accordance with claim 8, wherein the second treatment protocol step and the third protocol treatment step each are associated with a treatment protocol template that includes one or more parameter fields associated with one or more rules of the second or third treatment protocol step.

13. The method of claim 8, wherein the second or third protocol treatment steps specify a next clinical decision point in the same treatment protocol as the treatment protocol of the treatment protocol step associated with the generated treatment protocol template or specify a clinical decision point in a different treatment protocol.

14. Computer readable physical storage media having stored thereon computer readable instructions that when executed by a processor, cause a computing system to perform the method of claim 8.

15. A computer system comprising:
a first module configured to access a patient treatment protocol, the patient treatment protocol including a plurality of clinical decision points that include one or more rules that are to be complied with in order to successfully treat a patient using a medical device that is associated with the patient treatment protocol;
a treatment protocol template generation module configured to generate a treatment protocol template for each of the clinical decision points, each treatment protocol template specifying the one or more rules of the corresponding clinical decision point;
a next step logic module configured to determine a second protocol template that is to be accessed and followed when the one or more rules of a specific generated treatment protocol template are successfully passed and to determine a third protocol template that is to be accessed and followed when the one or more rules of the specific generated treatment protocol template are not successfully passed; and
one or more input modules for receiving information related to the specific generated treatment protocol template.

16. The computer system in accordance with claim 15, wherein the medical device is a ventilator.

17. The computing system in accordance with claim 15, further comprising:
an alarm module configured to provide an alarm when the received information is below a predetermined acceptable level.

18. The computing system in accordance with claim 15 further comprising:
a waveform generator configured to generate a real time waveform that looks at a mechanics of at least some of the received information.

19. The computing system in accordance with claim 15, further comprising:
a report module configured to track input received from user into the generated treatment protocol template and to report if there is a deviation from the rules specified in the generated treatment protocol template.

20. The computing system in accordance with claim 15, wherein the received information is at least one of patient information related to a patient connected to the medical device that is associated with the treatment protocol or information related to the medical device.
